(19) United States
(12) Patent Application Publication
(10) Pub. No.: US 2006/0153782 A1
(43) Pub. Date: Jul. 13, 2006

(54) Skin photoageing and actinic damage treatment

(75) Inventors: Graham Edmund Kelly, Northbridge (AU); Alan Husband, McMahons Point (AU); Cath Walker, Balmain (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Novogen Research Pty Ltd, North Ryde (AU)

(21) Appl. No.: 10/528,911

(22) PCT Filed: Sep. 23, 2003
(86) PCT No.: PCT/AU03/01265

§ 371 Date: Oct. 7, 2005

(30) Foreign Application Priority Data
Sep. 23, 2002 (AU) 2002951572
Jan. 21, 2003 (AU) 2003900236

(51) Int. Cl. A61K 8/49

(52) U.S. Cl. 424/59

(57) ABSTRACT

Use of equol, dehydroequol, and other isoflav-3-enes are described for the prevention and/or treatment of skin photoageing and actinic damage. Methods of treating these conditions are also described.

### FIELD OF THE INVENTION

The present invention relates to the use of equol and dehydroequol in particular, and compounds based on an isoflavonoid ring structure in general for the prevention and/or treatment of skin photoageing and actinic damage.

### BACKGROUND

DNA damage in skin cells is particularly important to human health because it can have major effects on skin appearance and well-being, in particular skin carcinogenesis. DNA damage occurs when the ultraviolet (UV) light component (particularly UV-B and UV-C) of sunlight passes through to the lower layers of the epidermis. In its passage through the epidermis, the UV irradiation causes mutations in the DNA strands in the genomes of all cells in the skin. Those mutations are known as pyrimidine dimers which normally are repaired automatically by specialist intra-nuclear enzymes such as endonucleases, with complete repair taking about 2-3 days. Repair involves the excision of the damaged segment and insertion of a new segment. DNA damage caused by UV-induced oxidative stress, which following a complex lengthy cascade resulting in the generation of reactive oxygen species (ROS), takes up to 3 days to occur.

This DNA damage has a number of potentially damaging consequences, particularly where the sunlight exposure is repeated and occurs over many years. These include a small proportion of dimers being mis-repaired, predisposing to mutagenic damage, in particular if the mis-repair occurs in important quality assurance genes such as p53. The accumulation of these mis-repaired genes over a lifetime believed to be a major predisposing factor to skin cancer.

The consequences of UV-induced DNA damage in skin, or other UV-induced skin damage may be associated with photoageing, actinic damage and carcinogenesis. These terms generally have the following meaning:
1. Photoageing refers to the process of accelerated ageing in sunlight-exposed skin. This embraces fine lines and wrinkles, freckles, yellowing of the skin, stretching, dilated capillaries (telangiectasis), cherry red spots (angiomas), and a dry complexion.
2. Actinic damage refers to pre-malignant or benign skin growths and embraces lesions such as solar keratoses or actinic keratoses.
3. Skin cancer refers to lesions with malignant potential and includes basal cell carcinoma, Bowen's disease (in situ squamous cell carcinoma), squamous cell carcinoma and melanoma.

The use of anti-inflammatory agents, skin rehydration, collagen injections, surgery and dermabrasion are just some of the many cosmetic products and procedures employed in attempts to redress the consequences of photoageing, and actinic damage.

A strategy that was able to promote DNA protection and/or repair would have several important benefits. First, by reducing the time to effect DNA repair, the pathological consequences would be reduced. Second, the repair process would be more efficient with less likelihood of mis-repairs occurring. The benefit of this strategy is confirmed by the use of topical administration of endonucleases in patients with the genetic disorder, xeroderma pigmentosus. Individuals with this condition fail to make endonucleases, the consequence of which is a high risk of malignant skin cancer and photoageing of skin following sunlight exposure. The application to the skin of these individuals of exogenous endonucleases significantly reduces the risk of these individuals to skin cancer and address photoageing. Thirdly, by increasing the production of free radical scavengers in the skin, DNA would be protected from oxidative stress lesions that form in response to UV exposure.

It has been speculated that certain compounds, including equol, may have the ability to prevent the onset of some symptoms of ageing in skin (U.S. Pat. No. 6,060,070, Gorbach).

The Gorbach patent is concerned with the natural process of ageing that is associated with all tissues in the body and may be associated with reduced estrogen function with advancing age. Lowered collagen content and reduced numbers of elastin fibres in skin as a consequence of falling estrogen levels are though to be the primary factors causing age-related wrinkles. Normal ageing is a distinctive entity to photoageing.

It has now been found by the applicants that compounds of the present invention, namely equol, dehydroequol and other isoflav-3-ene and isoflavan compounds, when applied to the skin or administered orally or parenterally, surprisingly promote repair of pyrimidine dimers and reduce oxidative stress lesions in skin. It was entirely unexpected that the compounds of the present invention promoted DNA repair, and even more surprising to find that they promoted DNA repair and protection, and could be used to prevent and/or treat skin photoageing and actinic damage.

In accordance with a first aspect of this invention there is provided use of equol, dehydroequol, or other isoflav-3-ene or isoflavan structures for the prevention and/or treatment of photoageing in skin subject to UV exposure. Photoageing includes lines, wrinkles, freckles, yellowing of skin, skin stretching, dilated capillaries, cherry red spots and dry complexion.

In another aspect of this invention there is provided use of the compounds of the invention in the prevention and/or treatment of actinic damage. Actinic damage includes solar keratoses or actinic keratoses.

In accordance with another aspect of this invention there is provided a method for the prevention and/or treatment of photoageing in skin subject to UV exposure which comprises administering to a subject a composition containing one or more of equol, dehydroequol, or other isoflav-3-ene, or isoflavan compounds in admixture with one or more acceptable carriers and/or excipients.

In accordance with another aspect of this invention there is provided a method for the prevention and/or treatment of actinic damage which comprises administering to a subject a composition containing one or more of equol, dehydroequol, or other isoflav-3-ene, or isoflavan compounds in admixture with one or more acceptable carriers and/or excipients.

Isoflav-3-ene and isoflavan compounds may be represented by the general formula (II) 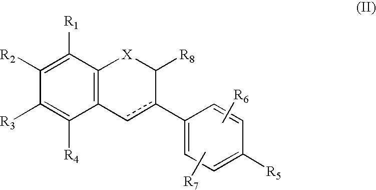
in which
R₁, R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₃ and R₄ are as previously defined, and R₁ and R₂ taken together with the carbon atoms to which they are attached form a five-membered ring selected from 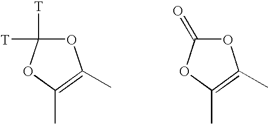
R₁ and R₄ are as previously defined, and R₂ and R₃ taken together with the carbon atoms to which they are attached form a five-membered ring selected from 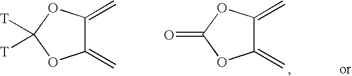
R₁ and R₂ are as previously defined, and R₃ and R₄ taken together with the carbon atoms to which they are attached form a five-membered ring selected from 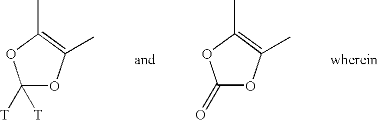
R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,
R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,
R₉ is alkyl, haloalkyl, aryl, arylalkyl, C(O)R₁₃ where R₁₃ is as previously defined, or Si(R₁₅)₃ where each R₁₅ is independently hydrogen, alkyl or aryl,
R₁₀ is hydrogen, alkyl, haloalkyl, amino, aryl, arylalkyl, an amino acid, alkylamino or dialkylamino,
R₁₁ is hydrogen, alkyl, arylalkyl, alkenyl, aryl, an amino acid, C(O)R₁₃ where R₁₃ is as previously defined, or CO₂R₁₄ where R₁₄ is as previously defined,
R₁₂ is hydrogen, alkyl or aryl, or
R₁₁ and R₁₂ taken together with the nitrogen to which they are attached comprise pyrrolidinyl or piperidinyl, the drawing [symbol] represents either a single bond or a double bond, preferably a double bond,
T is independently hydrogen, alkyl or aryl, and
X is O, NR₁₂ or S, preferably O,
   including pharmaceutically acceptable salts and derivatives thereof.

Preferably compounds of the formula II are equol and dehydroequol.

Most people, including children, teenagers, adults, and the elderly are exposed to UV exposure and sunlight. Indeed, sunlight provides the principal UV exposure experienced by skin. It is believed that most people would benefit from use of compounds of the present invention.

Compounds of the present invention prevent or treat photoageing and actinic damage. Further, compounds of the present invention promote both the rate and extent of DNA repair and protection in skin.

Compounds according to the present invention may be administered topically, orally or parenterally, or by other modes of administration.

Preferably, compositions containing one or more compounds according to the present invention are applied to the skin either before, at the time of, or after UV or sunlight exposure. For example, compositions may be in the form of a cream, including face cream or skin cream, lotion, cosmetic formulation and the like. For example, compounds of the present invention may be simply mixed, admixed, or blended with suitable carriers or bases to give compositions suitable for application to the skin.

Compounds of the formula II may be generally used in amounts from 20 μg to 500 mg/kg body weight of a subject. Topical compositions may contain compounds of the formula II on a w/w % basis of, for example, 0.01 to 60% w/w, with the remainder comprising carriers and/or excipients and/or standard components used in dermally acceptable compositions as are known in the art.

Compounds of the present invention have preventative and/or treatment applications as described herein. The compounds are preventative in that they lessen, inhibit, or generally prevent photoageing in skin subject to UV exposure and actinic damage. Compounds of the present invention are useful in the treatment of the aforementioned conditions in providing ameliorative outcomes once a subject experiences one or more of the conditions. The compounds of the present invention may be considered as both preventative and as a treatment of the aforementioned conditions in that they prevent or lessen photoageing, or actinic damage, or skin cancers, whilst at the same time treating the condition at hand.

The applicant has found that the compounds according to this invention promote DNA repair. The promotion of DNA repair may be by one or more of increasing the rate of repair of cyclobutane pyrimidine dimers (CPDs), promoting DNA repair by decreasing P53 expression, and/or by promoting the formation of metallothionein (MT). These effects may be responsible for the prevention and/or treatment of skin photoageing and actinic damage through promoting skin health and condition, and preventing skin cell damage.

The formation of CPD is considered to be an important lethal and mutagenic consequence of UVR exposure (Mitchell et al, 1989; Liardet et al, 2000). Animal models have demonstrated an inverse relationship between epidermal CPD repair and skin carcinogenesis (Young et al, 1996). The P53 protein (TP53) is expressed after DNA damage by UV irradiation. P53 is a transcription factor which blocks cellular progression from G1 to S phase, thus preventing replication of damaged DNA (Campbell et al, 1993). The P53 protein may act as a tumour promoting agent (Murphey et al, 2001).

This invention will be described with reference to the following, non-limiting examples.

### EXAMPLE 1

Equol was applied to the skin of five human volunteers immediately after and at 4 hours and 6 hours post-UV irradiation. Twenty-four hours after UV irradiation, MT production was measured. A control lotion was also used containing no equol. This experiment demonstrated that equol caused a statistically significant (P=0.469) elevation in the level of MT in the basal layer of irradiated skin (24 hour post-UV) when compared with unirradiated base line skin (pre-UVR). The vehicle itself did not statistically alter the level of MT in the basal layer of irradiated skin, when compared with unirradiated base line skin.

A reduction in skin wrinkling, capillary dilation and dry skin may also be observed.

### EXAMPLE 2

Cyclobutane Pyrimidine Dimers (CPD):

The formation of CPD's, which occurs immediately on UV exposure (Viv Reeve, pers comm) would be unaffected by any therapeutic agent applied post-UVR. However, the rate of repair of CPDs might be increased by equol. If this occurred, fewer CPDs in equol treated skin compared with the number in vehicle-only treated skin would be observed. 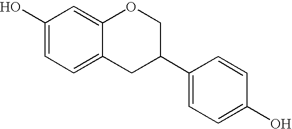
Equol (CAS No. 531-95-3)

There were few CPDs in the unirradiated skin of the human volunteer, who demonstrated the expected marked elevation 10 minutes after UV exposure. The human subject treated demonstrated a lower percentage of CPD+ve epidermal cells in equol treated skin.

Lower levels of CPD may be associated with preventing and/or treating lines, wrinkles, freckles, yellowing of skin, stretching of skin, dilated capillaries, cherry red spots, dry complexion, solar keratoses or actinic keratoses.

### EXAMPLE 3

Hairless mice treated with equol or dehydroequol either before or after chronic UV exposure show decreased skin thickness than non-treated mice. Increased skin thickness may be associated with wrinkles, capillary dilation in skin and skin dryness, as well as actinic damage.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The reference to any prior art in this specification is not, and should not be taken as an acknowledgment or any form of suggestion that that prior art forms part of the common general knowledge in Australia.

### REFERENCES

Campbell, C., Quinn, A. G., Angus, B., Farr, P. M. and Rees, J. L. (1993) “Wavelength specific patterns of p 53 induction in human skin following exposure to UV radiation” Cancer Research 52(12): 2697-9
Hanada, K., Baba, T., Hashimoto, I., Fukui, R. and Watanabe, S (1992) “Possible role of cutaneous metallothionein in protection against photo-oxiditative stress-epidermal localization and scavenging activity for superoxide and hydroxyl radicals” Photodermatology, Photoimmunology & Photomedicine 9(5): 209-13
Liardet, S., Scaletta, C., Panizzon, R., Hohlfeld, P., and Laurent-Applegate L. (2001) “Protection against pyrimidine dimers, p 53, and 8-hydroxy-2′-deoxyguaosine expressionin ultraviolet-irradiated human skin by sunscreens: Difference between UVB+UVA and UVA alone sunscreens” Journal of Investigative Dermatology 117: 1437-1441
Mitchell, D. L. and Nairn, R. S. (1989) “The biology of the (6-4) photoproduct” Photochemistry & Photobiology 49(6): 805-19
Murphey, R., Young, A. R., Wulf, H. C., Kulms, D. and Schwarz, T. (2001) “The molecular determinants of sunburn cell formation” Experimental Dermatology 10(3): 155-60
Young, A. R., Chadwick, C. A., Harrison, G. I., Hawk, J. J., Nikaido, O. and Potten, C. S. (1996) “The in situ repair kinetics of epidermal thymine dimers and 6-4 photoproducts in human skin types I and II” Journal of Investigative Dermatology 106(6): 1307-13

1. Use of compounds of the formula II for the prevention and/or treatment of skin photoageing or actinic damage of skin associated with UV exposure, wherein said compounds of the formula II comprise 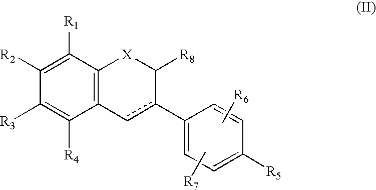
in which
R₁, R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₃ and R₄ are as previously defined, and R₁ and R₂ taken together with the carbon atoms to which they are attached form a five-membered ring selected from 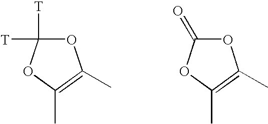
R₁ and R₄ are as previously defined, and R₂ and R₃ taken together with the carbon atoms to which they are attached form a five-membered ring selected from 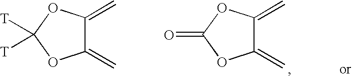
or
R₁ and R₂ are as previously defined, and R₃ and R₄ taken together with the carbon atoms to which they are attached form a five-membered ring selected from 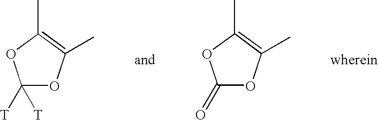
R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,
R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,
R₉ is alkyl, haloalkyl, aryl, arylalkyl, C(O)R₁₃ where R₁₃ is as previously defined, or Si(R₁₅)₃ where each R₁₅ is independently hydrogen, alkyl or aryl,
R₁₀ is hydrogen, alkyl, haloalkyl, amino, aryl, arylalkyl, an amino acid, alkylamino or dialkylamino,
R₁₁ is hydrogen, alkyl, arylalkyl, alkenyl, aryl, an amino acid, C(O)R₁₃ where R₁₃ is as previously defined, or CO₂R₁₄ where R₁₄ is as previously defined,
R₁₂ is hydrogen, alkyl or aryl, or
R₁₁ and R₁₂ taken together with the nitrogen to which they are attached comprise pyrrolidinyl or piperidinyl,
the drawing “ [symbol] ” represents either a single bond or a double bond, preferably a double bond,
T is independently hydrogen, alkyl or aryl, and
X is O, NR₁₂ or S, preferably O,
including pharmaceutically acceptable salts and derivatives thereof.

2. Use according to claim 1 for the prevention and/or treatment of skin photoageing selected from lines, wrinkles, freckles, yellowing of skin, skin stretching, dilated capillaries, cherry red spots and dry complexion.

3. Use according to claim 1 for the prevention and/or treatment of actinic damage selected from solar keratoses or actinic keratoses.

4. Use according to claim 1 wherein said compounds of the formula (II) comprise equol or dehydroequol.

5. A method for the prevention and/or treatment of skin photoageing or actinic damage of skin which comprises administering to a subject one or more compounds of the general formula (II) 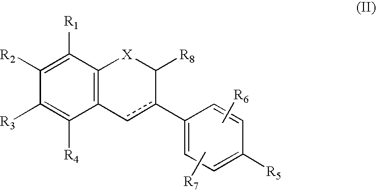
in which
R₁, R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₃ and R₄ are as previously defined, and R₁ and R₂ taken together with the carbon atoms to which they are attached form a five-membered ring selected from 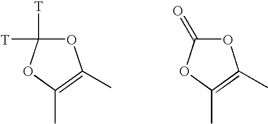
R₁ and R₄ are as previously defined, and R₂ and R₃ taken together with the carbon atoms to which they are attached form a five-membered ring selected from 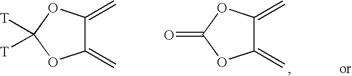
or
R₁ and R₂ are as previously defined, and R₃ and R₄ taken together with the carbon atoms to which they are attached form a five-membered ring selected from 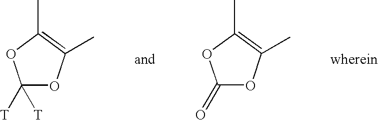
R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,
R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,
R₉ is alkyl, haloalkyl, aryl, arylalkyl, C(O)R₁₃ where R₁₃ is as previously defined, or Si(R₁₅)₃ where each R₁₅ is independently hydrogen, alkyl or aryl,
R₁₀ is hydrogen, alkyl, haloalkyl, amino, aryl, arylalkyl, an amino acid, alkylamino or dialkylamino,
R₁₁ is hydrogen, alkyl, arylalkyl, alkenyl, aryl, an amino acid, C(O)R₁₃ where R₁₃ is as previously defined, or CO₂R₁₄ where R₁₄ is as previously defined,
R₁₂ is hydrogen, alkyl or aryl, or
R₁₁ and R₁₂ taken together with the nitrogen to which they are attached comprise pyrrolidinyl or piperidinyl,
the drawing “ [symbol] ” represents either a single bond or a double bond, preferably a double bond,
T is independently hydrogen, alkyl or aryl, and
X is O, NR₁₂ or S, preferably O,
including pharmaceutically acceptable salts and derivatives thereof.

6. A method according to claim 5 wherein said one or more compounds of the formula (II) comprises equol and dehydroequol.

7. A method according to claim 5 which is a method for the prevention and/or treatment of skin photoageing selected from lines, wrinkles, freckles, yellowing of skin, skin stretching, dilated capillaries, cherry red spots and dry complexion.

8. A method according to claim 5 which is a method for the prevention and/or treatment of actinic damage selected from solar keratoses or actinic keratoses.

9. A method according to claim 5 wherein said one or more compounds of the formula (II) are administered orally, parenterally or topically, before and/or after skin exposure.
